(19) United States
(12) Patent Application Publication
(10) Pub. No.: US 2026/0194498 A1
(43) Pub. Date: Jul. 9, 2026

(54) NOVEL ELECTRONIC NOSE APPARATUS FOR DETECTING PORK SCENT AND THE METHOD THEREOF

(71) Applicant: Ting-Wei Hsu, Taipei City (TW)

(72) Inventors: Ting-Wei Hsu, Taipei City (TW); Tung-Yueh Lin, Taipei City (TW)

(21) Appl. No.: 19/008,905

(22) Filed: Jan. 3, 2025

(51) Int. Cl. G01N 30/72; G01N 30/02; G01N 30/86; G01N 30/88; G01N 33/00

(52) U.S. Cl. CPC G01N 30/7206; G01N 30/86; G01N 33/0047; G01N 2030/025; G01N 2030/884

(57) ABSTRACT

Provided are a novel electronic nose apparatus for detecting pork scent and a method thereof. The apparatus includes an air suction hood arranged above a baggage conveyor belt, an air suction tube and a gas analyzer connected to the air suction hood through the air suction tube. The air suction hood conducts real-time sampling of gas around an item to be inspected on the baggage conveyor belt, and the sampled gas is fed into the gas analyzer, and the gas analyzer detects whether the sampled gas contains 2,3-octanedione contained, to confirm whether the item to be inspected contains the targeted contraband. This invention is advantageous in that a targeted compound in an extremely small amount can be analyzed by instrumental analysis, thereby improving sensitivity and specificity of inspection, and preventing illegal entry and smuggling of contraband, e.g., meat products or drugs, reducing work burden of detection dogs, or even replacing their task.

### BACKGROUND OF THE INVENTION

### 1. Field of the Invention

The present invention relates to a novel electronic nose apparatus for detecting pork scent and the method thereof, wherein particularly the scent molecule (2,3-octanedione) of the targeted compound can be analyzed through an apparatus, thereby the fatigue of the detection dogs as performing tasks frequently can be alleviated.

### 2. The Prior Arts

African swine fever is a highly contagious and severe disease in pigs, with the virus able to persist in meat for extended periods (100 days in chilled pork, and 1000 days in frozen pork). The World Organisation for Animal Health (WOAH) currently designates it as a notifiable disease, as a result, countries have strengthened border control measures.

Although the X-ray machines at airports or customs are highly sensitive to metal detection, it can not identify whether the baggage contains pork and the products thereof. Therefore, the scent-based inspections of baggage currently rely on working dogs with keen olfactory senses (e.g., detection dogs), such as Labrador Retrievers or Beagles.

In addition, to protect the ecological environment, agricultural safety and public safety, border controls in various countries also use detection dogs to check whether passengers carry other contraband, such as:
(1) agricultural products, including fresh fruits, vegetables, seeds, plants, flowers, and so on, which may carry pests or pathogens;
(2) meat and the products thereof, including raw meat, cooked food, preserved foods, which may carry animal and plant diseases, such as foot-and-mouth disease or avian influenza;
(3) dairy products and eggs, which may carry animal diseases;
(4) illegal wildlife and the products thereof, such as ivory, rhino horn, rare birds, turtle shells, and other internationally protected wildlife;
(5) drugs and explosives.

However, such inspection method is prone to miss certain items, and may also cause the detection dogs to become fatigued from overwork.

Therefore, there is a need to develop a novel electronic nose technology to assist or replace detection dogs in performing the hectic inspection tasks.

### SUMMARY OF THE INVENTION

To solve the aforementioned problems, an object of the present invention is to provide a novel electronic nose apparatus for detecting pork scent and the method thereof, to avoid over-relying on the scent-based inspections by detection dogs at airports and customs, which results in missed contraband detections when the detection dogs are unable to handle the workload.

Therefore, to achieve the aforementioned object, the present invention provides a novel electronic nose apparatus for detecting pork scent, comprising: an air suction hood arranged above a baggage conveyor belt, an air suction tube and a gas analyzer,
wherein the air suction tube connects the air suction hood to the gas analyzer,
the air suction hood conducts real-time sampling of gas around an item to be inspected on the baggage conveyor belt, and the sampled gas is fed through the air suction tube into the gas analyzer, and
the gas analyzer detects whether the sampled gas contains a specific scent molecule contained in a targeted contraband, to confirm whether the item to be inspected contains the targeted contraband,
wherein the targeted contraband is selected from pork and products thereof, and the specific scent molecule is 2,3-octanedione.

To achieve the aforementioned object, the present invention also provides a method for detecting pork scent applicable for a novel electronic nose, comprising:
a sampling step, conducting real-time sampling of gas around an item to be inspected on the baggage conveyor belt;
a sample injecting step, feeding the sampled gas into the gas analyzer; and
an analyzing step, detecting whether the sampled gas contains a specific scent molecule with the gas analyzer, to confirm whether the item to be inspected contains the targeted contraband,
wherein the targeted contraband is selected from pork and products thereof, and the specific scent molecule is 2,3-octanedione.

In one embodiment of the present invention, the gas analyzer is a real-time gas chromatography-tandem mass spectrometer, which consists of a gas chromatography mass spectrometer and a tandem mass spectrometer.

In one embodiment of the present invention, a spectrum of the specific scent molecule is pre-established in the gas analyzer, and when the sampled gas is fed into the gas analyzer, the gas analyzer analyzes a spectrum of the sampled gas, and compares the spectrum of the sampled gas with the pre-established spectrum of the specific scent molecule, to confirm whether the item to be inspected contains the targeted contraband.

In one embodiment of the present invention, when the spectrum of the sampled gas matches the spectrum of the specific scent molecule, it is determined that the item to be inspected contains the targeted contraband; and when the spectrum of the sampled gas does not match the spectrum of the specific scent molecule, it is determined that the item to be inspected does not contain the targeted contraband.

The present invention is advantageous in that a more sensitive apparatus is utilized to detect and analyze the gas molecules at airports or customs, to determine whether the item to be inspected contains the targeted contraband (e.g., pork and the products thereof), which also ensures sufficient rest for overworked customs officers and detection dogs.

The effect of the present invention is not limited to those mentioned above, and those skilled in the art may clearly understand other effects not mentioned from the description of claims.

### BRIEF DESCRIPTION OF THE DRAWINGS

FIG. 1 shows the mass spectrum analysis of 2,3-octanedione.

FIG. 2 is the view of a novel electronic nose apparatus for detecting pork scent according to the present invention.

FIG. 3 is a flowchart of a method for detecting pork scent applicable for a novel electronic nose according to the present invention.

### DETAILED DESCRIPTION OF THE PREFERRED EMBODIMENT

In light of the aforementioned problems, the inventors of the present invention developed a novel electronic nose apparatus for detecting pork scent and the method thereof, wherein a detector is used to specifically analyze the scent composition in the baggage of passengers to identify whether it contains the targeted contraband.

Specifically, to enhance the convenience of daily life, the inventors developed a highly sensitive detection apparatus designed to specifically identify the scent molecules of the contraband (e.g., pork and the products thereof), which is arranged above the baggage conveyor belt to replace the detection dogs that have been working long hours at airports or customs. Therefore, the border control can be conducted around the clock, while hardworking officers and detection dogs can get sufficient rest.

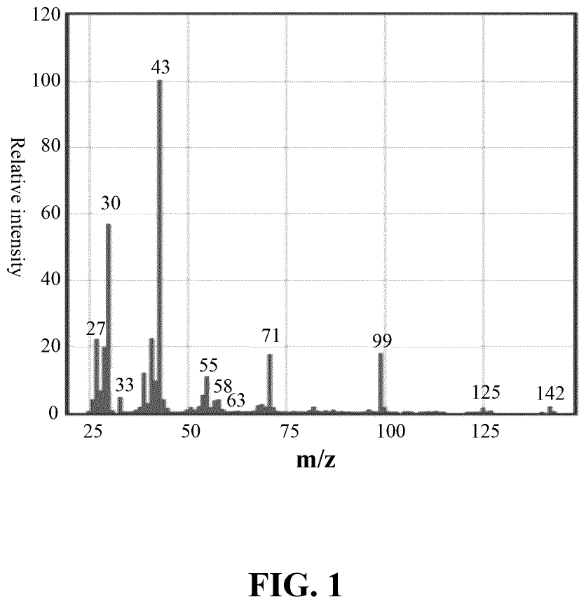

In particular, different meat, food or other contrabands carry different scent molecules. Taking pork and the products thereof for example, one of the scent molecules of pork is 2,3-octanedione. As shown in FIG. 1, which shows the mass spectrum analysis of 2,3-octanedione. Therefore, inspecting whether the baggage contains pork and the products thereof and even other contrabands by instrumental analysis is available.

To analyze the scent molecules (e.g., 2,3-octanedione) contained in the contraband accurately and sensitively, in the method of the present invention, a real-time gas chromatography-tandem mass spectrometer (Real-time GC-MS/MS) is used, which consists of a gas chromatography mass spectrometer (GC-MS) and a tandem mass spectrometer, and is suitable for analyzing volatile compounds with low boiling points. Compared to general commercially available electric noses, a real-time gas chromatography-tandem mass spectrometer can analyze a targeted compound in an extremely small amount, thereby greatly improving the sensitivity and specificity of inspection, as shown in Table 1 below.

TABLE 1
Comparison table of commercially available electronic noses
and the method for detecting pork scent applicable for a
novel electronic nose according to the present invention.

| Inspection | General commercially | Method applicable for the novel |
|---|---|---|
| of scent | available electric | electronic nose of the present |
| molecules | noses | invention |
| Features | The analysis is | Quantitative analysis can be |
| | performed for only | performed by using chromatographic |
| | certain scent | columns to separate individual |
| | molecules, and is | substances and then utilizing a |
| | mostly qualitative | tandem mass spectrometer to detect |
| | analysis. | low-concentration gas molecules. |
| Price | Low | Higher |
| | (About 50,000 to | (About 3,000,000 NTD) |
| | 120,000 NTD) | |
| Appli- | Easily purchasable | Suitable for official organizations |
| cation | by individuals | such as airports, seaports, or research |
| charac- | | institutions. |
| teristics | | |

In the present invention, an air suction hood is installed above the baggage conveyor belt of airports or customs along with an air suction tube to immediately feed air into a gas analyzer (e.g., real-time gas chromatography-tandem mass spectrometer, real-time GC-MS/MS), to analyze spectrums of different sampled gases.

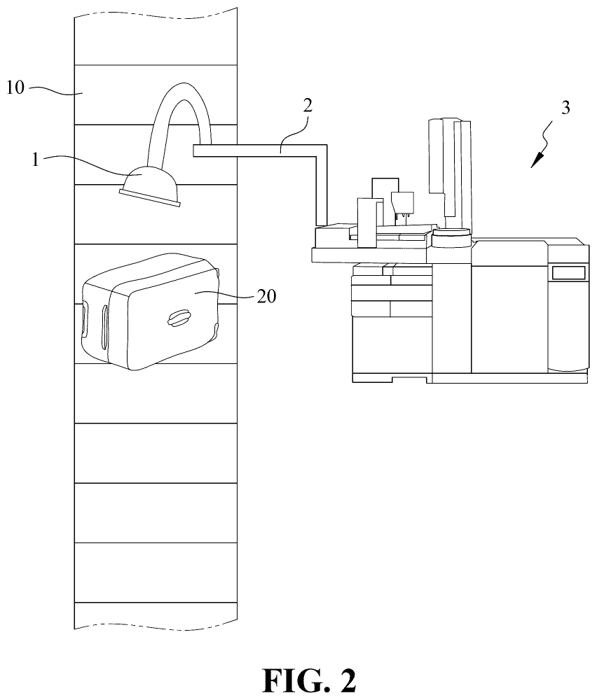

Therefore, as shown in FIG. 2, the present invention provides a novel electronic nose apparatus for detecting pork scent, comprising an air suction hood 1 arranged above a baggage conveyor belt 10 (e.g., a baggage conveyor belt of airports and customs), an air suction tube 2, and a gas analyzer 3.

Wherein, the air suction tube 2 connects the air suction hood 1 to the gas analyzer 3.

The air suction hood 1 conducts real-time sampling of gas around an item to be inspected 20 on the baggage conveyor belt 10, and the sampled gas is fed through the air suction tube 2 into the gas analyzer 3.

Then, the gas analyzer 3 detects whether the sampled gas contains a specific scent molecule contained in a targeted contraband, to confirm whether the item to be inspected 20 contains the targeted contraband.

In the present invention, the targeted contraband may be selected from pork and the products thereof, and the specific scent molecule may be 2,3-octanedione.

The gas analyzer 3 may be a real-time gas chromatography-tandem mass spectrometer (Real-time GC-MS/MS), which consists of a gas chromatography mass spectrometer (GC-MS) and a tandem mass spectrometer.

For example, in the case where the targeted contraband is selected from pork and the products thereof, a spectrum of the specific scent molecule (e.g., 2,3-octanedione) contained in pork and the products thereof may be pre-established in the gas analyzer 3; and subsequently, when the sampled gas is fed into the gas analyzer 3, the gas analyzer 3 analyzes a spectrum of the sampled gas, and compares the spectrum of the sampled gas with the pre-established spectrum of the specific scent molecule, when the two spectrums match, it is determined that the item to be inspected 20 contains the targeted contraband; and

On the other hand, when the spectrum of the sampled gas does not match the spectrum of the specific scent molecule, it is determined that the item to be inspected 20 does not contain the targeted contraband.

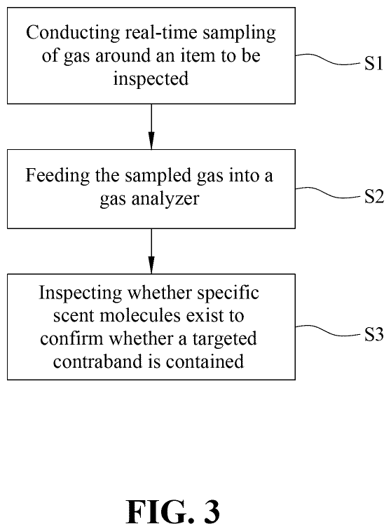

In addition, as shown in FIG. 3, the present invention also provides a method for detecting pork scent applicable for a novel electronic nose, comprising:
a sampling step S1, conducting real-time sampling of gas around an item to be inspected on the baggage conveyor belt;
a sample injecting step S2, feeding the sampled gas into the gas analyzer; and
an analyzing step S3: detecting whether the sampled gas contains a specific scent molecule contained in a targeted contraband with the gas analyzer, to confirm whether the item to be inspected contains the targeted contraband.

In the present invention, the targeted contraband may be selected from pork and the products thereof, and the specific scent molecule may be 2,3-octanedione.

In particular, the analyzing step S3 may be conducted by the following method: a spectrum of the specific scent molecule contained in targeted contraband may be pre-established in the gas analyzer, and subsequently, when the sampled gas is fed into the gas analyzer, the gas analyzer analyzes a spectrum of the sampled gas, and compares the spectrum of the sampled gas with the pre-established spectrum of the specific scent molecule, when the two spectrums match, it is determined that the item to be inspected contains the targeted contraband.

On the other hand, when the spectrum of the sampled gas does not match the spectrum of the specific scent molecule, it is determined that the item to be inspected does not contain the targeted contraband.

Different from commercially available electric noses, since the real-time gas chromatography-tandem mass spectrometer used in the present invention has higher sensitivity, and may analyze a wide variety of different items (e.g., food), obtain different spectrums and establish databases, while one of the scent molecules of pork is 2,3-octanedione, therefore, by analyzing this scent molecule, it is possible to quickly identify whether the baggage contains contrabands (e.g., pork and the products thereof).

The greatest advantage of the present invention is that the electronic noses may be installed near the outlet of the baggage conveyor belt, eliminating the need for scent-based inspections by detection dogs when passengers retrieve their baggage, which may reduce the work burden of the detection dogs, and even avoid the case of missed detections.

Another advantage of the present invention is that no other country has utilized instrumental analysis to replace detection dogs at airports or customs, comparing to this, the present invention adopts a real-time gas chromatography-tandem mass spectrometer, which may analyze a targeted compound in an extremely small amount, thereby greatly improving the sensitivity and specificity of inspection. In addition to the application in inspections of pork and the products thereof, applications in establishing more spectrums of other types of contrabands (e.g., drugs) for big data analysis are available, thereby preventing carrying or smuggling of contraband (e.g., meat, drugs) all-round, and replacing the role of detection dogs (e.g., drug detection dogs) at airports.

Moreover, such concept also can be applied in detecting scent molecules of other contrabands, such as various drugs.

Moreover, since it is necessary to analyze the scent molecules sensitively, the gas chromatography mass spectrometer used in the present invention is not quite expensive and more suitable for application in official institutions, such as airports, seaports, government agencies, and key administrative units.

The technical features of any embodiments of the present invention may be combined with those of other embodiments of the present invention, provided there are no contradictions.

What is claimed is:

1. A novel electronic nose apparatus for detecting pork scent, comprising: an air suction hood arranged above a baggage conveyor belt; an air suction tube; and a gas analyzer,
wherein the air suction tube connects the air suction hood to the gas analyzer, the air suction hood conducts real-time sampling of gas around an item to be inspected on the baggage conveyor belt, and the sampled gas is fed through the air suction tube into the gas analyzer, and
the gas analyzer detects whether the sampled gas contains a specific scent molecule contained in a targeted contraband, to confirm whether the item to be inspected contains the targeted contraband,
wherein the targeted contraband is selected from pork and products thereof, and the specific scent molecule is 2,3-octanedione.

2. The novel electronic nose apparatus according to claim 1, wherein the gas analyzer is a real-time gas chromatography-tandem mass spectrometer, which consists of a gas chromatography mass spectrometer and a tandem mass spectrometer.

3. The novel electronic nose apparatus according to claim 1, wherein a spectrum of the specific scent molecule is pre-established in the gas analyzer, and
when the sampled gas is fed into the gas analyzer, the gas analyzer analyzes a spectrum of the sampled gas, and compares the spectrum of the sampled gas with the pre-established spectrum of the specific scent molecule, to confirm whether the item to be inspected contains the targeted contraband.

4. The novel electronic nose apparatus according to claim 3, wherein
when the spectrum of the sampled gas matches the spectrum of the specific scent molecule, it is determined that the item to be inspected contains the targeted contraband; and
when the spectrum of the sampled gas does not match the spectrum of the specific scent molecule, it is determined that the item to be inspected does not contain the targeted contraband.

5. A method for detecting pork scent applicable for a novel electronic nose comprising:
a sampling step, conducting real-time sampling of gas around an item to be inspected on the baggage conveyor belt;
a sample injecting step, feeding the sampled gas into the gas analyzer; and
an analyzing step, detecting whether the sampled gas contains a specific scent molecule with the gas analyzer, to confirm whether the item to be inspected contains the targeted contraband,
wherein the targeted contraband is selected from pork and products thereof, and the specific scent molecule is 2,3-octanedione.

6. The method for detecting pork scent according to claim 5, wherein the gas analyzer is a real-time gas chromatography-tandem mass spectrometer, which consists of a gas chromatography mass spectrometer and a tandem mass spectrometer.

7. The method for detecting pork scent according to claim 5, wherein a spectrum of the specific scent molecule is pre-established in the gas analyzer, and
in the analyzing step, the gas analyzer analyzes a spectrum of the sampled gas, and compare the spectrum of the sampled gas with the pre-established spectrum of the specific scent molecule, to confirm whether the item to be inspected contains the targeted contraband.

8. The method for detecting pork scent according to claim 7, wherein in the analyzing step,
when the spectrum of the sampled gas matches the spectrum of the specific scent molecule, it is determined that the item to be inspected contains the targeted contraband; and
when the spectrum of the sampled gas does not match the spectrum of the specific scent molecule, it is determined that the item to be inspected does not contain the targeted contraband.
